(19) United States
(12) Patent Application Publication
(10) Pub. No.: US 2025/0269348 A1
(43) Pub. Date: Aug. 28, 2025

(54) PHOTOCATALYTIC REDUCTION OF CARBON DIOXIDE IN A DUAL OPTICAL-FIBER PHOTOCATALYTIC SYSTEM

(71) Applicants: Yen-Jung Lai, Chandler, AZ (US); Tzu-Heng Wang, Tainan City (TW); Paul K. Westerhoff, Scottsdale, AZ (US); Bruce E. Rittmann, Tempe, AZ (US)

(72) Inventors: Yen-Jung Lai, Chandler, AZ (US); Tzu-Heng Wang, Tainan City (TW); Paul K. Westerhoff, Scottsdale, AZ (US); Bruce E. Rittmann, Tempe, AZ (US)

(21) Appl. No.: 19/065,574

(22) Filed: Feb. 27, 2025

Related U.S. Application Data
(60) Provisional application No. 63/558,229, filed on Feb. 27, 2024.

(51) Int. Cl. B01J 19/24; B01J 31/16; B01J 35/39; C07C 51/00

(52) U.S. Cl. CPC B01J 19/2475; B01J 31/1691; B01J 35/39; C07C 51/00; B01J 2219/00711; B01J 2219/0072; B01J 2235/15; B01J 2531/842; C07C 2531/22

(57) ABSTRACT

A reactor for photocatalytic reduction of carbon dioxide includes a first reactor, a second reactor, and a light source. The first reactor includes a multiplicity of hollow-fiber membranes. The first reactor is configured to solubilize gaseous carbon dioxide to yield aqueous carbon dioxide. The second reactor is in fluid communication with the first reactor and includes a side-emitting polymeric optical fiber with a photocatalytic coating. The second reactor is configured to accept the aqueous carbon dioxide and the photocatalytic coating includes an iron-based metal-organic framework. The light source is optically coupled to the side-emitting polymeric optical fiber. Reducing carbon dioxide includes solubilizing gaseous carbon dioxide to yield aqueous carbon dioxide, contacting a side-emitting polymeric optical fiber including a photocatalytic coating with the aqueous carbon dioxide, and providing visible radiation to the side-emitting polymeric optical fiber, thereby reducing the aqueous carbon dioxide. The photocatalytic coating includes an iron-based metal organic framework.

### CROSS-REFERENCE TO RELATED APPLICATION

This application claims the benefit of U.S. Patent Application No. 63/558,229 filed on Feb. 27, 2024, which is incorporated herein by reference in its entirety.

### STATEMENT OF GOVERNMENT SUPPORT

This invention was made with government support under 1449500 awarded by the National Science Foundation. The government has certain rights in the invention.

### TECHNICAL FIELD

This invention relates to a dual optical-fiber photocatalytic system including a side-emitting optical fiber coated with an iron-based metal-organic framework catalyst for photocatalytic reduction of carbon dioxide.

### BACKGROUND

Carbon dioxide (CO₂) can be converted into valuable organic chemicals using light irradiation and photocatalysis. While photocatalysis has the capability to harness the entire solar spectrum, including ultraviolet, visible, and infrared wavelengths, visible light constitutes about half of the solar spectrum.

### SUMMARY

This disclosure describes a dual fiber reactor system that combines a polymeric optical fiber (POF) coated with an iron-containing metal-organic framework (MOF) catalyst with hollow-fiber membranes for photocatalytic reduction of carbon dioxide (CO₂) (e.g., to formic acid (HCOOH)). The photocatalytic POF reactor platform described herein demonstrates high efficiency production of formic acid by coating an iron-based metal-organic framework (Fe-MOF) onto side-emitting POFs and using hollow-fiber membranes to deliver bubble-free aqueous carbon dioxide The solubilization of gaseous carbon dioxide yields aqueous carbon dioxide. Bubble-free delivery of aqueous carbon dioxide allows efficient transfer by eliminating CO₂ off-gassing associated with bubble sparging. The photocatalyst-Fe-MOF with amine-group (—NH₂) functional groups-improves dissolved inorganic carbon absorption.

In one example, the dual-fiber system has a CO₂-to-HCOOH conversion rate of 116±1.2 mM h⁻¹ g⁻¹, with a quantum efficiency for photocatalytic HCOOH generation of 12%. The conversion efficiency of CO₂-to-HCOOH is about 22%. The product selectivity of CO₂-to-HCOOH is about 99%. Due at least in part to the dual efficiencies of bubble-free CO₂ delivery and high quantum efficiency, the dual-fiber system has an energy consumption of only 0.60±0.05 k Wh mole⁻¹. This dual-fiber design enables efficient CO₂ valorization without use of platinum group metals or rare earth elements.

In a first general aspect, a reactor system for photocatalytic reduction of carbon dioxide includes a first reactor, a second reactor, and a light source. The first reactor includes a multiplicity of hollow-fiber membranes, wherein the first reactor is configured to solubilize gaseous carbon dioxide to yield an aqueous carbon dioxide. The second reactor can be in fluid communication with the first reactor. The second reactor includes a side-emitting polymeric optical fiber with a photocatalytic coating. The second reactor can be configured to accept the aqueous carbon dioxide. In some cases, the photocatalytic coating includes an iron-based metal-organic framework. The light source can be optically coupled to the side-emitting polymeric optical fiber.

Implementations of the first general aspect may include one or more of the following features. In some cases, the first reactor is configured to yield bubble-free aqueous carbon dioxide. The photocatalytic coating can be porous. In some certain implementations, the iron-based metal-organic framework includes amine moieties. In some cases, the light source is configured to irradiate the side-emitting polymeric optical fiber with visible light. The photocatalytic coating can be configured to improve light-harvesting and facilitate reduction of the carbon dioxide. In some examples, the reactor system is configured to produce formic acid via reduction of carbon dioxide. In certain implementations, a loading of the photocatalytic coating on the side-emitting polymeric optical fiber is in a range of about 1 μg cm⁻² to about 35 μg cm⁻². The multiplicity of hollow-fiber membranes can be permeable to carbon dioxide.

In a second general aspect, reducing carbon dioxide includes solubilizing gaseous carbon dioxide to yield an aqueous carbon dioxide, contacting a side-emitting polymeric optical fiber including a photocatalytic coating with the aqueous carbon dioxide, wherein the photocatalytic coating includes an iron-based metal-organic framework, and providing visible radiation to the side-emitting polymeric optical fiber, thereby reducing the aqueous carbon dioxide.

Implementations of the second general aspect may include one or more of the following features. In some cases, the aqueous carbon dioxide is free of bubbles. In certain implementations, reducing the aqueous carbon dioxide yields formic acid.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 is a schematic illustration of photocatalytic formic acid (HCOOH) production in the dual fiber reactor system, including polymeric optical fibers coated with NH₂-MIL-101(Fe) photocatalyst and hollow-fiber membranes for CO₂ delivery.

FIG. 2 shows side-emitted light intensity along a 20-cm POF for different mass loadings of multiple coating of photocatalyst layers of 5 g NH₂-MIL-101(Fe) L⁻¹.

FIG. 3 shows light side-emission and harvesting efficiencies of pristine and modified POFs. The side emission (I_{S}) and light utilization (I_{U}) values were changed with mass loadings of NH₂-MIL101(Fe) (μg cm⁻²) exposed to 440 nm light emitting diode (LED) light (3 W) irradiation.

FIG. 4A shows photocatalytic CO₂ reduction followed zero-order kinetics for HCOOH evolution with various NH₂-MIL-101(Fe) on POF loadings. FIG. 4B shows the normalized HCOOH production rates peaked at the surface loading of 34 μg cm⁻² NH₂-MIL-101(Fe). Irradiation was 3 W with a 440-nm LED light.

FIG. 5 shows HCOOH-generation of POF—NH₂-MIL-101(Fe), POF-MIL-101(Fe), and a photocatalytic NH₂-MIL-101(Fe) slurry reaction. Irradiation was from a 3-W LED at 440 nm.

FIG. 6A shows generation curves for HCOOH, CO, H₂, and O₂ for POF—NH₂-MIL-101(Fe) (34 μg cm⁻²). FIG. 6B shows carbon mass balance on products of the dual fiber system using the optimal POF—NH₂-MIL-101(Fe) (34 μg cm⁻²) with 440-nm LED (3 W) and a CO₂ flux at 0.39 mg-C min⁻¹ from the hollow fiber membranes.

FIG. 7A shows carbon conversion for photocatalytic CO₂ reduction using 34 μg cm⁻² NH₂-MIL-101(Fe) for different CO_{2(g)}-supply pressures, 3 mM potassium iodide (KI), irradiation at 3 W with a 440-nm LED. FIG. 7B shows the carbon mass balance at the end of 2 hours.

### DETAILED DESCRIPTION

This disclosure describes a dual fiber reactor system that combines a polymeric optical fiber (POF) coated with an iron-containing metal-organic framework (MOF) catalyst with hollow-fiber membranes for photocatalytic reduction of carbon dioxide (CO₂). MOFs, a class of compounds with metal ions or clusters coordinated with organic ligands to form one-, two-, or three-dimensional structures, are responsive to visible light. For simultaneous light delivery and CO₂ interactions, catalysts have been evaluated in slurry and/or coated flat-plate systems with a high-power light source that irradiates the entire liquid reactor in which CO₂ (g) is delivered via bubbling. Systems described herein show at least a 10-fold improvement in light conversion efficiency of CO₂ to a valorized product.

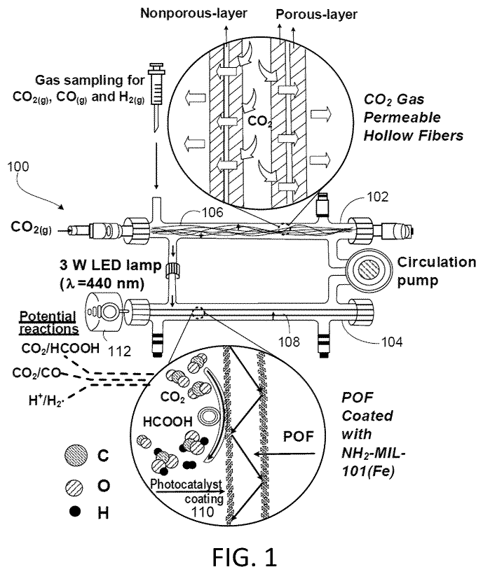

FIG. 1 depicts a reaction system 100 for photocatalytic reduction of carbon dioxide. The system 100 includes a first reactor 102 and a second reactor 104. The first reactor 102 includes a multiplicity of hollow-fiber membranes 106. The first reactor 102 is configured to solubilize gaseous carbon dioxide (e.g., in water) to yield aqueous carbon dioxide. The second reactor 104 includes a side-emitting polymeric optical fiber 108 with a photocatalytic coating 110. The second reactor 104 is in fluid communication with the first reactor 102. The second reactor 104 is configured to accept the aqueous carbon dioxide. The photocatalytic coating 110 includes an iron-based metal organic framework. A light source 112 is optically coupled to the side-emitting polymeric optical fiber 108.

The dual-fiber reactor combines a POF onto which the MOF-based catalyst is coated and hollow-fiber membranes to deliver CO₂ gas without bubble formation. Bubble-free delivery of dissolved CO_{2(aq)} through hollow fiber membranes enables close to 100% efficient CO_{2(g)} transfer by eliminating CO₂ off-gassing associated with bubble sparging. Dual-fiber systems described herein improve light-harvesting efficiency as well as interactions among molecular CO_{2(aq)}, photo-generated electrons (e⁻), and protons (H⁺), and facilitate catalyst reuse.

This disclosure describes integration of nanostructured NH₂-MIL-101(Fe) onto the POF to improve the light-harvesting and CO₂ adsorption ability, including quantification of photocatalytic CO₂-to-formic acid (HCOOH) evolution and production stability within the process and evaluation of the quantum efficiency and selectivity for carbon-based products. As used herein, “formic acid” generally refers to formic acid and ionic forms thereof (e.g., formate).

Through coupling a POF coated with NH₂-MIL-101(Fe) for catalyzed CO₂ reduction and hollow-fiber membranes for efficient CO₂ delivery, dual-fiber systems as described allow for a rate of CO₂-to-HCOOH production as high as 116 mM h⁻¹ g⁻¹. This rate is 25-fold greater than values generally observed in a photocatalytic slurry system. The optimized condition achieves a 12% quantum efficiency, which is 18-fold higher than the quantum efficiency generally obtained from photocatalytic NH₂-MIL-101(Fe) in a slurry reactor. The optimal NH₂-MIL-101(Fe) mass loading on the POF for formic acid production corresponds to the loading that achieves the highest side emission and light utilization percentage values. This correspondence reinforces that one benefit of the optical fiber is superior light delivery to the photocatalysts, at least in part because coating MOF photocatalysts on the POF surface enables continuous exposure to light without relying on light penetration through the water column or reactor wall (e.g., as in slurry systems). In addition, the side emission (Iₛ) and light utilization (I_{U}) of the POFs can be correlated with CO₂ conversion, at least in part because an optical fiber with high side-emission allows more photons to be emitted across the cladding along the length of fibers, leading to higher quantum efficiency for CO₂ reduction. The hollow-fiber membranes allow effective bubble-free CO₂ delivery that enabled simple and robust control of CO₂ mass transport that can be synchronized with the CO₂ flux needed to reduce CO₂ to HCOOH. The resulting selectivity to HCOOH is about 99%.

Scaling up the dual-fiber reactor can be optimized by increasing the specific surface areas of the MOF-coated POF and the hollow-fiber membranes. The former can increase the volumetric production rate, while the latter can match the CO₂ delivery rate to the CO₂ reduction rate while also maintaining high selectivity for HCOOH.

### Examples

### Materials and Methods

Iron (III) chloride hexahydrate (FeCl₃·6H₂O, ≥99.5%), N, N-dimethylformamide (DMF, ≥99%), methanol (≥99.8%), isopropanol (99%), and potassium iodide (KI, ≥99.5%) were purchased from Sigma-Aldrich Co. 2-aminoterephthalic acid (NH₂—H₂BDC, 99%) and Nafion (5% w/w in water and 1-propanol) were obtained from Thermo Scientific (USA). All chemicals were of analytical grade and were used directly without purification. Poly(methyl methacrylate)-polymeric optical fibers (POF, CK-120, 3.0 mm diameter, refractive index of 1.49) were obtained from Industrial Fiber Optics (AZ, USA).

Formic acid (HCOOH), CO_{(g)} and H_{2(g)} measurements: 1 mL samples were filtered using a 0.2 μm polyvinylidene fluoride filter and then analyzed by ion chromatography (Metrohm 930 Compact IC). The ion chromatography had a Metrosep A Supp 5-250/4.0 column and was run with an eluent of 1 mM sodium bicarbonate (NaHCO₃) plus 3.2 mM sodium carbonate (Na₂CO₃) at a flow rate of 0.7 mL min⁻¹. Gaseous products (CO_{(g)} and H_{2(g)}) were analyzed with a gas chromatograph (Shimadzu GC 2010) equipped with a thermal conductivity detector held at 200° C. The packed column (Carboxen 1010 PLOT, 30 m length) having a 0.53 μm inner diameter was operated isothermally at 230° C. and with ultra-high purity argon (>99.999%) as the carrier gas at a flow rate of 40 mL min⁻¹.

The morphology and elemental distribution of pristine and modified POF surfaces were analyzed using a scanning electron microscope (JEOL JXA-8530F) coupled with energy dispersive X-ray diffraction and transmission electron microscope at 120 kV. The crystallographic plane was assessed by X-ray diffraction via Malvern PANalytical Aeris X-ray Diffractometer for POFs with Cu Kα radiation (λ=1.5406 Å) at a voltage of 40 kV and a current of 40 mA. X-ray photoelectron spectrometry ESCA PHI 1600 using an AL dual anode of X-ray with a photon energy of 1486.6 cV was utilized to identify the chemical elements of POF-MIL-101(Fe). A Fourier transform infrared spectrometer (Bruker, IFS66V/S) recorded chemical bonding in the wavenumber range of 700-4000 cm⁻¹ resolution. The absorption wavelength and valence band maximum of MIL-101(Fe) were measured by an ultraviolet-visible spectrophotometer (U-4100 Hitachi) and ultraviolet photoelectron spectroscopy with an ultra-violet He source. N₂ adsorption-desorption curves, specific surface area, and pore size distribution were obtained by Brunauer-Emmett-Teller specific surface area analyzer (BET, ASAP 2000). The Fe loading on the POFs was determined by an inductively coupled plasma-mass spectrometer (Perkin Elmer Inc., NexION 2000) followed by dissolving the POF-MIL-101(Fe) using the isopropanol (99% purity, Sigma-Aldrich).

### Example 1. Fabrication of POF—NH₂-MIL-101(Fe)

Poly (methyl methacrylate)-polymeric optical fibers were pre-treated by polishing of the cut surface. Scanning electron microscope shows that the cut surfaces on both sides of bare and modified POFs had uniform surfaces that reproducibly transmitted light into the lumen of the fiber.

NH₂-MIL-101(Fe) was prepared according to known procedures with some modification. Briefly, 1.35 g FeCl₃·6H₂O (5 mmol) and 0.45 g NH₂—H₂BDC (2.5 mmol) were dissolved into 35 mL of dimethylformamide with continuous stirring for 1.5 hours at room temperature to form a homogenous solution. The mixed solution was put into an autoclaved 50-mL Teflon stainless steel reactor (BAOSHISHAN, China) at 110° C. for 20 hours via hydrothermal processing. After cooling, the dark brown mixture was washed four times each with deionized water and methanol through vacuum filtration. The brown powders were dried at 100° C. in an oven for 24 hours to obtain the NH₂-MIL-101(Fe).

A well-dispersed slurry with concentration from 1 to 10 g L⁻¹ was produced by adding fixed NH₂-MIL-101(Fe) masses into 30 mL of isopropanol containing a 15% of 5% Nafion® polymer solution; this mixture was stirred and sonicated for 2 hours in an ice bath. The POFs were dip-coated into the NH₂-MIL-101(Fe) solution for two seconds, followed by oven-drying for 2 minutes at 60° C., rinsing with deionized water, and drying for 12 hours at 60° C. The modified POFs were decorated with NH₂-MIL-101(Fe) using dip-coating twice into NH₂-MIL-101(Fe) slurries, the concentration ranging between 0-10 g L⁻¹. MOFs were well affixed to the POF surface in a semi-porous coating, similar to other nanoparticle-coated POFs. Mass loadings (μg cm⁻²) of iron (Fe) ranging from 10 to 56 μg cm⁻² were quantified by inductively coupled plasma-mass spectrometry.

X-ray diffraction displays distinct peaks at 5.03°, 8.36°, and 8.96° 20, affirming high crystallinity. Fourier transform infrared spectroscopy detected absorption peaks at 1337, 1440-1600, 1626, 3456, and 3373 cm⁻¹, which correspond to C—N, O—C—O, the amine group with asymmetrical/symmetrical stretching vibration, σ (N—H), and ν (C—N), respectively. Fourier transform infrared spectroscopy identified functional groups on the surface of NH₂-MIL-101(Fe) prove that the polymerization reaction of MOF synthesis achieved the desired hybrid metal/organic nanostructure. The full-scan X-ray photoelectron spectrometry spectrum of NH₂-MIL-101(Fe) provides insights into the elemental composition, with peaks for C, N, O, and Fe. ultraviolet-visible spectroscopy and ultraviolet photoelectron spectroscopy measurements show optical properties that reveal significant absorption in the ultraviolet-visible spectrum, a 1.75 eV band gap, and efficient visible-light-driven photocatalysis with a corresponding band gap of 1.01 V (valence band vs NHE) and −0.74 V (conduction band vs NHE).

An ultraviolet-visible spectrum of pristine NH₂-MIL-101 (Fe) photocatalysts, calculated bandgap spectrum, and valence-band spectrum illustrate the mechanistic rationale behind the NH₂-MIL-101(Fe) photocatalyst's ability to generate HCOOH from carbon dioxide (CO₂) (−0.61 V vs NHE) and H₂ from H₂O (−0.41 V vs normal hydrogen electrode (NHE)). X-ray diffraction and X-ray photoelectron spectrometry analyses confirm the fabrication of the desired MOFs, showcasing the self-assembly of iron, organic linkers, and ammonium. Scanning electron microscopy analysis reveals the NH₂-MIL-101(Fe) with a hexagonal micro-spindle crystal, featuring uniform particles with a 250-nm diameter.

The morphology and particle size of pristine NH₂-MIL-101(Fe) and POF—NH₂-MIL-101(Fe) were characterized by scanning electron microscopy and energy dispersive X-ray diffraction. Electron microscope images with elemental distributions for the exterior surface of uncoated POF and POF-MIL-101(Fe) show the surface of bare POF contained only O, F, and C. The two elements form the basic composition of the polymethyl methacrylate polymer with a fluorinated cladding layer. The POF coated with NH₂-MIL-101(Fe) indicated a uniform surface coverage of Fe and N. Results of Fe quantification by inductively coupled plasma-mass spectrometry showed a range from zero on the bare fiber to 34 μg cm⁻² for the MOF coated POF (e.g., POF—NH₂-MIL-101(Fe)). Characterization of multiple fibers provided reproducible results. Overall, the dip-coating process produced a POF—NH₂-MIL-101(Fe) that could be used in the dual-fiber reactor system, as shown in FIG. 1.

### Example 2. Fabrication of Hollow-Fiber Membrane Bundles

To deliver bubble-free CO₂ into the reactor, 8 composite hollow-fiber membranes (200 μm inner and 280 μm outer diameter with 15 cm length; Model MHF 200TL, Mitsubishi Rayon, Japan) were bundled together at both ends using polyurethane tubing (¼ quarter inch diameter, Surethane NSF-51, ATP, USA). Briefly, hollow-fiber membranes were bundled together at the both ends using polyurethane tubing (¼ quarter inch diameter, Surethane NSF-51, ATP, USA) as the fiber-bundle adapter. A bundle was built using 8 hollow-fiber membranes of ˜15 cm in length. Resins with low and high viscosity obtained from Polymer Composites, Inc. (CA, USA) were applied to glue the fiber bundle. Low-viscosity resins were employed to encase the fiber bundle, and the potting method eliminated fiber pinched during the cutting. Prior to use, fiber bundles underwent a leak test at 10 psig (corresponding to 24.7 psia or 1.68 atm) N₂ gas by submerging the fiber bundle in the deionized water. For quality control of bundle fabrication, the gas flow of tested bundles (Alicat, US) was verified; gas flow was 318±59 cm³ min⁻¹ obtained at the open end of the hollow-fibers with 10 psig of applied N₂ gas. The CO₂ flux (mg-C min⁻¹) was obtained using an 8-fiber membrane bundle at desired pressure supply from 2 to 10 psig with 100% CO₂ (Matheson, Phoenix) and conducted in an independently duplicate setting. The net inorganic-carbon increase within a 1 hour time window was quantified by the Total Organic Carbon analyzer (Teledyne Tekmar Lotix, USA), and the transfer rates of 0.15 to 0.39 mg-C min⁻¹ corresponded for 2 to 10 psig, respectively.

### Example 3. Light Utilization Efficiency of POF—NH₂-MIL-101(Fe)

Photon irradiance was measured at multiple locations for the optical fiber, including total light input, the refraction of light side-emission, and light transmission. Light was delivered into the POF using a monochromatic light emitting diode (LED) light (3.0 W, λ=440 nm, Uxcell, China) with an applied voltage of 5.4 V and a current of 0.55 A. Light-energy intensity (μW cm⁻²) was measured every 2-cm apart by a spectrophotoradiometer (Avantes AvaSpec-2048 L, Louisville, CO) equipped with a with a 600 μm jacketed silica fiber optic cable (FC-UV600-1-ME-SR) and a Cosine Corrector (CosC) attached to collect light using a radiometer (CC-UV/VIS/NIR-8 MM). The side-emission measurement was conducted by placing the spectrophotoradiometer tip perpendicular to the optical fiber surface. All measurements were performed using triplicate fibers.

Intensity denotes the directional energy density of a light ray (μW/cm²), calculated by multiplying the energy density by the velocity vector of the light. Power, on the other hand, refers to the radiant or intensity flux received on a surface, or the cumulative energy of each photon collected on a surface. Therefore, Equation 1 embodies a conservation of power based on intensity (μW/cm²) and the areas irradiated:

$${P}_{Abs ( bare PMMA POF )} = {P}_{0} - {P}_{T} - {P}_{S} - {P}_{U} \qquad ( 1 )$$

where P₀ and P_{T} is the power (μW) entering and exiting (at 20-cm distal end) the POF. P₀ and P_{T} is calculated from measured irradiance entering (I₀) and exiting (IT) the 20 cm long POF multiplied by the POF cross-sectional area (πr²). P_{S} is the scattering power due at least in part to side emission on the outer POF surface where the photocatalyst is located. P_{S} is calculated by integrating the area under the curve of side-emitted light scattering intensity (I_{S,x}) measured every 2 cm along the 20-cm fiber length (x from 0 to 20 cm) using Origin 2018 version b.9.5.0.193, and then accounting for the outer surface area (2 πr) of the fiber where the catalyst is located; the POF diameter is 3 mm (r=0.15 cm). The percentages of power and intensity for the entire POF length are provided by Eq (2) through (4):

$${P}_{Abs ( bare PMMA POF )} ( \% ) = \frac{{P}_{0} - {P}_{T} - {P}_{S} - {P}_{U}}{{P}_{0}} \times 100 \% \qquad ( 2 )$$

$${I}_{S} ( \% ) \approx \frac{{P}_{s}}{{P}_{0}} \times 100 \% = \frac{2 π r {\int }_{x = 0 cm}^{x = 20 cm} {I}_{3 , x} dx}{{P}_{0}} \times 100 \% \qquad ( 3 )$$

$${I}_{U} ( \% ) \approx \frac{{P}_{U}}{{P}_{0}} \times 100 \% = \frac{{P}_{0} - {P}_{Abs} - {P}_{S} - {P}_{T}}{{P}_{0}} \times 100 \% \qquad ( 4 )$$

where some energy is lost due at least in part to absorbance within the PMMA material of the POF (P_{Abs(bare PMMA POF)} (%)), while other energy is scattered or utilized by the catalyst. Catalysts are activated by the intensity of light, and irradiance signifies the radiant flux over a real surface (μW/cm²). Therefore the percentage of irradiance is considered, as conservation relies on the intensity of each wave, for the amount of side-emitted light (I_{S} (%)) or light utilized by the photocatalyst (I_{U} (%)), which are approximately equivalent to the ratios of P_{S} or P_{U} to P₀, respectively.

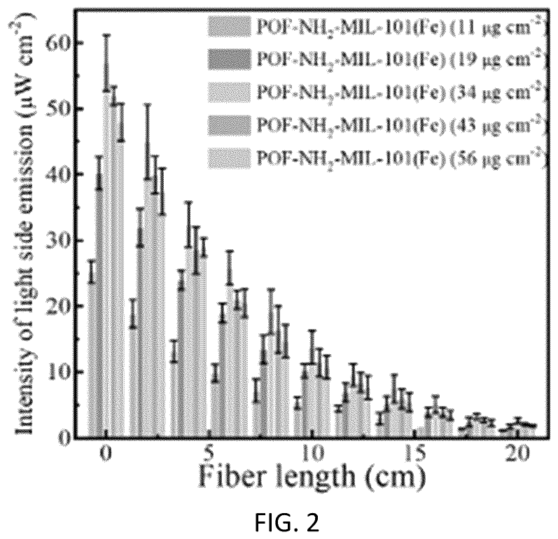

Photocatalytic activity capable of converting aqueous inorganic carbon to organic carbon is driven by the delivery of visible light to the surface of the POF on which the NH₂-MIL-101(Fe) is embedded. The bare fiber side emits light because of refractive index differences between the polymethyl methacrylate cladding. When the MOF is applied to the fiber surface, it enables the absorption and utilization of light by NH₂-MIL-101(Fe). FIG. 2 shows the intensities of side-emitted 440-nm light along a 20 cm length of POF with different NH₂-MIL-101(Fe) loadings. Bare POFs exhibited a side emission of 1.1 μW cm⁻² at the proximal end, and it exponentially declined along the 20 cm length to 1 μW cm⁻² at the distal end. For a POF loaded with 10 μg cm⁻² NH₂-MIL-101(Fe), the side-emission light intensity increased to 21 μW cm⁻² at the proximal end and 1 μW cm⁻² at the distal end. A higher NH₂-MIL-101 loadings (e.g., 34 μg cm⁻²) led to increased side-emitted light, with values reaching 5.7 μW cm⁻² at the proximal end and 3 μW cm⁻² at the distal end. The observed enhancements in side emission can be attributed to alterations in the refractive index and/or the introduction of additional Rayleigh scattering at the POF-water interface resulting from the NH₂-MIL-101(Fe) coating.

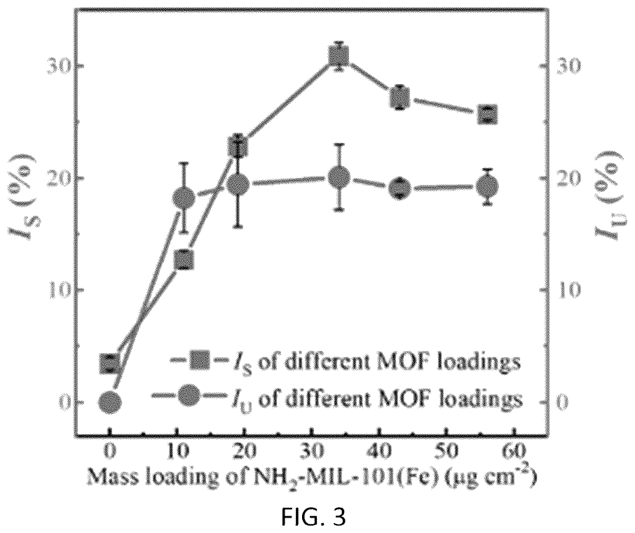

The highest side emission Is (%) and the optimal light utilization (It), calculated by Eq. (4) and depicted in FIG. 3, indicate that a mass loading of 34 μg cm⁻² NH₂-MIL-101(Fe) yielded 31% Is and 21% I_{U}. Notably, the trends in I_{S} and I_{U} consistently showed that excessive photocatalyst loading compromised both metrics. The alignment of I_{S} and I_{U} results underscores that an optimal mass loading maximized side emissions, thus contributing to increased photon utilization. The highest values for I_{S} and I_{U} were attained with the 3-cycle coating, resulting from the deposition of 34 μg cm⁻² NH₂-MIL-101(Fe). Therefore, 34 μg cm⁻² NH₂-MIL-101(Fe) was deemed an acceptable loading for light utilization.

### Example 4. Integrated Dual-Fiber Reactor System for Photocatalytic HCOOH Generation

Photocatalytic HCOOH production was evaluated in a photocatalytic dual-fiber reactor that combined the modified POF and hollow fiber membranes in two parallel and connected glass columns (0.8 cm diameter; 18 cm reactor length), each having 10-mL total volume, as shown in FIG. 1. One 20 cm-long bare or coated POF (e.g., 20.7 cm² of outer surface area) and a ˜15 cm-long hollow fiber membrane bundle were mounted in separate glass columns and connected using Versilon™. A-60-N tubing (Masterflex, MFLX06404-25). Ultrapure water (18.2 Ωcm, pH ˜7.1) in the column was purged with N₂ gas for 2 hours to remove dissolved oxygen before placing the water into the reactor. The reactor was continuously mixed by circulating water through the reactor system at a flow rate of 130 mL min⁻¹ (Master Flex pump, model 7520-40, Cole-Parmer Instrument Company, USA). The experiment had a 2-hour duration, and 1-mL liquid aliquots were collected from the reactor every 30 minutes (the reaction volume was reduced ˜8% after all samplings) and analyzed for pH (e.g., maintained at ˜3.8) and HCOOH_{(aq)}. pH was measured using a calibrated Lab 860 pH meter (Schott, Germany). Gas samples were collected from the headspace/gas bag with a 1-mL gas chromatography syringe and analyzed for CO_{(g)} and H_{2(g)}. For selected experiments, potassium iodide (10 mM, KI) as an electron donor (electron-hole scavenger) was added to the initial de-oxygenated water to give 3 mM KI as the final concentration; adding KI was used to gain understanding of hole scavenging on photocatalytic CO₂ reduction.

Formic acid was quantified by ion chromatography. 1-mL samples were filtered using a 0.2-μm polyvinylidene fluoride filter prior to ion chromatography analysis (IC 930, Metrohm). The ion chromatography had a Metrosep A Supp 5-250/4.0 column and was run with an eluent of 1-mM sodium bicarbonate (NaHCO₃) plus 3.2 pm sodium carbonate (Na₂CO₃) at a flow rate of 0.7 mL min⁻¹. Gaseous products (CO_{(g)}, CO_{2(g)} and H_{2(g)}) were analyzed with a gas chromatograph (Shimadzu GC 2010) equipped with a thermal conductivity detector held at 200° C. The packed column (Carboxen 1010 PLOT, 30 m length) having a 0.53-μm inner diameter was operated isothermally at 230° C. and with ultra-high purity argon (>99.999%) as the carrier gas at a flow rate of 40 mL min⁻¹. The gas volume in the Teflon bag was measured by a 60-mL gas-tight syringe. The concentrations of gas samples were utilized to calculate each value in grams using the Ideal Gas Equation.

The quantum efficiency (Φ_{QE}) for photocatalytic HCOOH generation was calculated as follows:

$${Φ}_{QE} ( \% ) = \frac{{C}_{HCOOH}}{{mol}_{photons}} \times 100 \% \qquad ( 5 )$$

where C_{HCOOH} is the number of moles of HCOOH generated, and molₚₕₒₜₒₙₛ is the moles of photons launched into the fiber.

The selectivity of CO₂ reduction to produce different carbon-based products considered two products (HCOOH and CO) based upon the following reduction half-reactions (6) and (7) vs Normal Hydrogen Electrode (NHE):

$$\begin{matrix}{CO}_{2} + 2 {H}^{+} + 2 {e}^{-} \to HCOOH & {E}^{0} = - 0.61 V vs . NHE\end{matrix} \qquad ( 6 )$$

$$\begin{matrix}{CO}_{2} + 2 {H}^{+} + 2 {e}^{-} \to CO & {E}^{0} = - 0.53 V vs . NHE\end{matrix} \qquad ( 7 )$$

The total carbon delivered to the system was calculated using equation (8):

$$Total carbon ( TC , g ) = ( {CO}_{2} flux ( C , {gh}^{- 1} ) \times duration ( h ) \qquad ( 8 )$$

where CO₂ flux (C·g h⁻¹) was obtained using an 8-fiber membrane bundle at the specified pressure. Additionally, the gaseous samples were quantified for their concentrations with gas chromatography. No other carbon-containing compounds were identified by HPLC (Shimadzu, Japan) equipped with an Aminex HPX-87H column. All species for the carbon mass balance species were converted to the unit of grams C. The mass of aqueous CO₂ at the end of the 2 hour experiments was computed by the difference (all terms are in g C): C—CO_{2(aq)}=Total C delivered (from the 2 hour CO₂ delivery capacity)−C-formate (from ion chromatography concentration multiplied by the liquid volume)−C—CO_{2(g)}/CO_{(g)} (from the headspace CO₂ concentration by gas chromatography multiplied by the gas volume).

The conversion efficiency (%) for photocatalytic CO₂ reduction was also quantified for HCOOH and CO using the following relationships:

$$HCOOH conversion ( \% ) = \frac{produced {HCOH}_{( aq )} ( gC )}{Total carbon ( gC )} \times 100 \% \qquad ( 9 )$$

$$CO conversion ( \% ) = \frac{produced {CO}_{( g )} ( gC )}{Total carbon ( gC )} \times 100 \% \qquad ( 10 )$$

The energy consumption of photocatalytic CO₂-to-HCOOH (kWh/mole_{HCOOH}) was computed from:

$$Energy consumption ( kWh {mole}^{- 1} ) = \frac{kW \times t}{m} \qquad ( 11 )$$

where kW is the light energy utilized by the photocatalytic layers, t is the time (hour), and m is the mole of HCOOH.

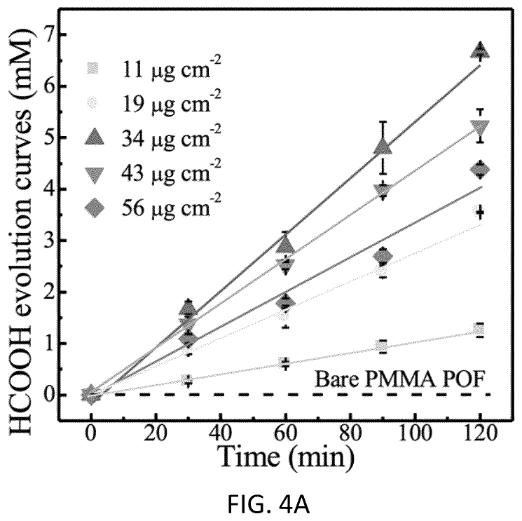

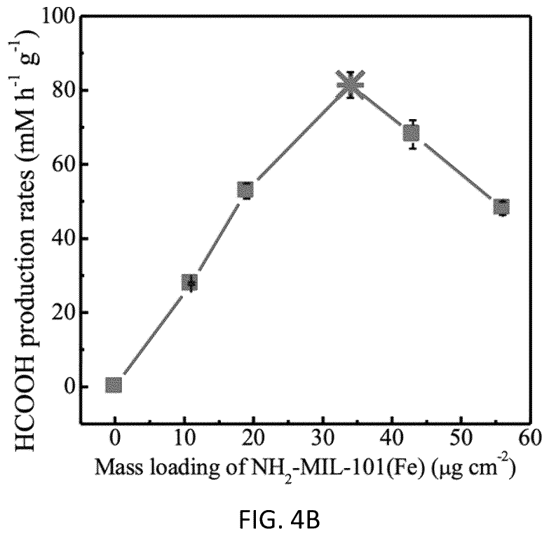

The dual-fiber reactor successfully generated high yields of HCOOH. FIGS. 4A and 4B show that the photocatalytic generation of HCOOH, CO, and H₂ occurred only for MOF-coated POFs; no other carbon products were identified. FIG. 4A illustrates the rapid formation of formic acid, with rates varying according to MOF loadings. The kinetics of formic acid production were well represented by pseudo-zero order kinetics. The normalized k values in FIG. 4B reveal maximum photocatalytic activity for the 34 μg cm⁻² mass loading of POF—NH₂-MIL-101(Fe), which is consistent with the highest values for side emission and light utilization shown in FIGS. 2 and 3. The maximum formic-acid-production rate reached 82 mM h⁻¹ g⁻¹, approximately twice as high as rates observed for the mass loading of 56 μg cm⁻² NH₂-MIL-101(Fe) or the loading of 39 μg cm⁻² without R—NH₂ decorating (e.g., MIL-101(Fe)). Thus, the same mass loading (34 μg cm⁻²) that optimized for light utilization also resulted in the maximum rate of formic-acid production.

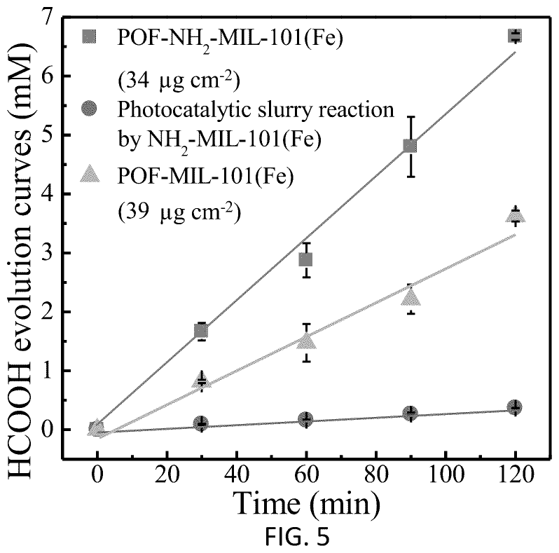

The maximum photocatalytic activity of the POF—NH₂-MIL-101(Fe) was 18-fold higher than the rate (˜5 mM h⁻¹g⁻¹) for a slurry containing NH₂-MIL-101(Fe) catalyst (e.g., the same catalyst weight coated on the optical fiber) under identical reactor configuration and irradiation conditions, as shown in FIG. 5. The slurry approach, where complete mixing occurred, resulted in a continuous movement of photocatalysts in and out of zones where the photons were delivered into the water. The optimized activity of POF—NH₂-MIL-101(Fe) was about 1.8-fold higher than the rate obtained from loading of 39 μg cm⁻² without R—NH₂ decorating (e.g., MIL-101(Fe)), indicating that the amine groups improved CO_{2(aq)} capture, thereby enhancing the adsorption of the carbonyl group (C═O) and facilitating the photocatalytic reduction. In contrast, MIL-101(Fe) catalysts lacking the —NH₂ modification showed inferior performance.

The net effect of the enhanced and consistent irradiation of the MOF on the POF led to the dual-fiber system achieving a remarkably high quantum efficiency. The quantum efficiency was 12% for the POF with optimal loading condition, which surpassed the quantum efficiency obtained from photocatalytic slurry from other photocatalysis (e.g., Au/TiO₂, ZnS, or Cu₂O) by 1.2- to 60-fold. Notably, the energy consumption with the optimal condition was only 0.6 k Wh mole⁻¹_{HCOOH}, which was about several magnitudes lower than photocatalytic CO₂ reduction in a slurry for other catalysts. Higher energy-conversion efficiencies were achieved through the dual efficiencies of CO₂ delivery via the gas permeable membrane, which prevents losses of CO₂ from water, and the high quantum yield achieved using the POF platform.

The POF with the optimal MOF loading had good reusability for CO₂ reduction. Consistent formic acid generation occurred over five sequential tests, each having a 2 hour duration. The calculated k values for HCOOH production were similar for each cycle (p≥0.05) documents the reusability of NH₂-MIL-101(Fe).

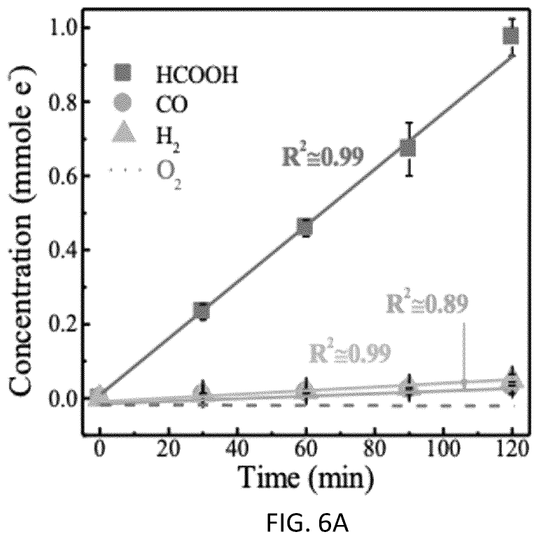

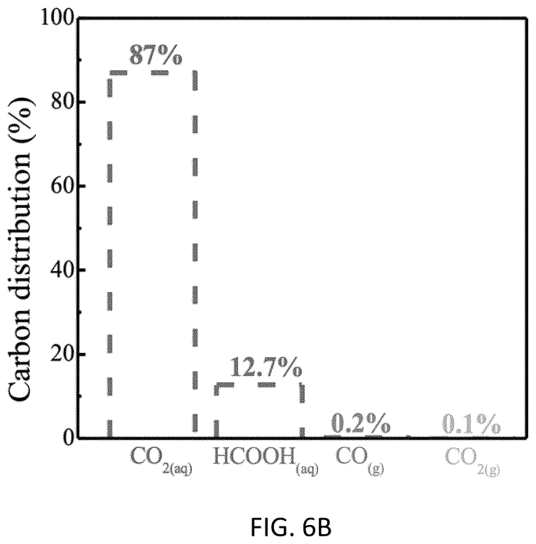

Formic acid was the dominant product, although other products (e.g., CO and H₂) were generated during photocatalytic CO₂ reduction. As shown in FIG. 6A, for the optimal mass loading (34 μg cm⁻²), the production curves of CO and H₂ were linear, reaching maximum concentrations of 1.4×10⁻² and 2.5×10⁻² mmole, respectively. As the conductive-to-valence band gap of NH₂-MIL-101(Fe) was not compatible with water oxidation potential, O₂ evolution did not occur during the photocatalysis. On an electron basis (mmol electrons per Equations (6) and (7)), the optimal fiber system was 34- and 19-fold more selective toward HCOOH than towards CO and H₂, respectively. FIG. 6B presents the carbon mass balance and shows that 12.7% of the CO₂ transferred from the membranes was routed to HCOOH, with CO only 0.2%; CO_{2(g)} and CO_{2(aq)} accounted for 87.0% and 0.1% of the total input carbon. Thus, the CO₂-supply rate (0.39 mg-C min⁻¹) slightly exceeded the photocatalytic CO₂-reduction capacity.

To enhance the conversion of CO₂ to HCOOH, the CO₂-supply pressure was varied within the range of 2 to 10 psig, which gave delivery capacities ranging from 0.15 to 0.39 mg-C min⁻¹. Also, experiments were performed in the presence of KI, an electron-hole scavenger, which should reduce the oxidation of formic acids by electron holes. The introduction of KI enhanced the HCOOH-production rate, increasing it from 82 to 116 mM h⁻¹ g⁻¹, as shown in FIGS. 4A and 6A.

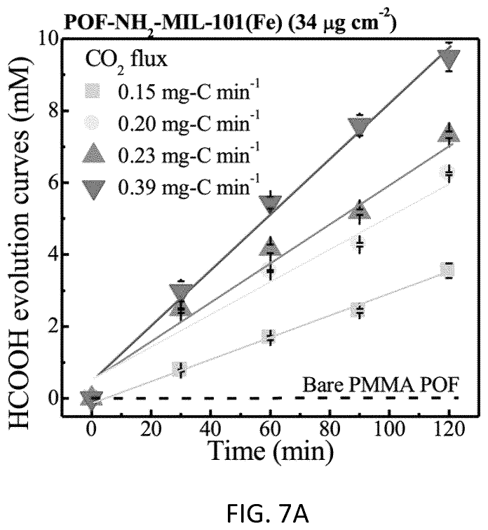

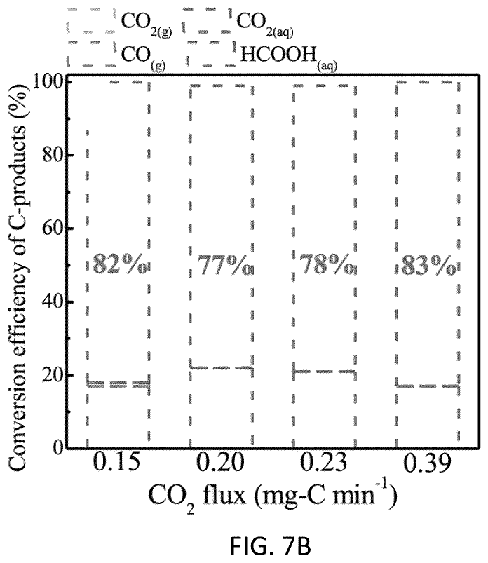

FIG. 7A shows that that the highest HCOOH-production rate (116 mM h⁻¹ g⁻¹; k=3.5×10⁻⁴ mM s⁻¹) was with the 10-psig CO₂ gas pressure. Notably, this rate is >2.7-fold higher than the value operated at 2 psig (k=1.9×10⁻⁴ mM s⁻¹). CO_{2(aq)} concentration was a key factor for the formic acid-production rate, and no formic acid was generated without CO₂ supply. Carbon transfer efficiency affects how much CO_{2(g)} must be supplied to attain a given CO_{2(aq)} concentration. In general, commercial spargers can achieve 20-40% carbon transfer efficiency, while hollow-fiber membranes can approach 100% carbon transfer efficiency for the medium pH near or above neutral. Bubbles formed by spargers can have the added disadvantage of disrupting the catalytic reaction by dispersing the light.

FIG. 7B reveals that the highest carbon-conversion efficiency (23%) of CO₂ to HCOOH was obtained with 3 psig CO₂ (0.2 mg-C min⁻¹), and the efficiency dropped to 17% at 10 psig (0.39 mg-C min⁻¹). As the water did not have a buffer to maintain the pH, a low pH 3.8-4.0 developed during CO₂(g) delivery. The low pH led to CO₂ off-gassing that lowered the carbon-transfer efficiency.

Through coupling a POF coated with NH₂-MIL-101(Fe) for catalyzed CO₂ reduction and hollow-fiber membranes for efficient CO₂ delivery, the dual-fiber system gave high-rate CO₂-to-HCOOH production, reaching as high as 116 mM h⁻¹ g⁻¹. This rate is 25-fold greater than any corresponding value observed in a photocatalytic slurry system. The optimized condition achieved a 16% quantum efficiency, which was >80-fold higher than the quantum efficiency obtained from photocatalytic NH₂-MIL-101(Fe) in a slurry reactor. The optimal NH₂-MIL-101(Fe) mass loading on the POF for formic acid production corresponded to the loading that achieved the highest side emission and light utilization. This correspondence reinforces that a benefit of the optical fiber is superior light delivery to the photocatalysts, because coating MOF photocatalysts on the POF surface enabled continuous exposure to light without relying on light penetration through the water column or reactor wall, as in slurry systems. In addition, the side emission and light utilization of POFs were correlated with CO₂ conversion, because an optical fiber with high side-emission allows more photons to be emitted across the cladding along the length of fibers, which led to higher quantum efficiency for CO₂ reduction. The hollow-fiber membranes provided effective bubble-free CO₂ delivery that enabled simple and robust control of CO₂ mass transport that could be synchronized with the CO₂ flux needed to reduce CO₂ to HCOOH; the selectivity to HCOOH was up to 99%.

Scaling up the dual-fiber reactor can be optimized by increasing the specific surface areas of the MOF-coated POF and the hollow-fiber membranes. The former can increase the volumetric production rate, while the latter can match the CO₂-delivery rate to the CO₂-reduction rate while also maintaining high selectivity for HCOOH. Another strategy for improving performance includes providing a pH buffer that does not allow the pH to decline, which allows CO₂ off-gassing.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A reactor system for photocatalytic reduction of carbon dioxide, the system comprising:
a first reactor comprising a multiplicity of hollow-fiber membranes, wherein the first reactor is configured to solubilize gaseous carbon dioxide to yield an aqueous carbon dioxide;
a second reactor in fluid communication with the first reactor and comprising a side-emitting polymeric optical fiber with a photocatalytic coating, wherein the second reactor is configured to accept the aqueous carbon dioxide and the photocatalytic coating comprises an iron-based metal-organic framework; and
a light source optically coupled to the side-emitting polymeric optical fiber.

2. The reactor system of claim 1, wherein the first reactor is configured to yield bubble-free aqueous carbon dioxide.

3. The reactor system of claim 1, wherein the photocatalytic coating is porous.

4. The reactor system of claim 1, wherein the iron-based metal-organic framework comprises amine moieties.

5. The reactor system of claim 1, wherein the light source is configured to irradiate the side-emitting polymeric optical fiber with visible light.

6. The reactor system of claim 1, wherein the photocatalytic coating is configured to improve light-harvesting and facilitate reduction of the carbon dioxide.

7. The reactor system of claim 1, wherein the reactor system is configured to produce formic acid via reduction of carbon dioxide.

8. The reactor system of claim 1, wherein a loading of the photocatalytic coating on the side-emitting polymeric optical fiber is in a range of about 1 μg cm⁻² to about 35 μg cm⁻².

9. The reactor system of claim 1, wherein the multiplicity of hollow-fiber membranes are permeable to carbon dioxide.

10. A method of reducing carbon dioxide, the method comprising:
solubilizing gaseous carbon dioxide to yield an aqueous carbon dioxide;
contacting a side-emitting polymeric optical fiber comprising a photocatalytic coating with the aqueous carbon dioxide, wherein the photocatalytic coating comprises an iron-based metal-organic framework; and
providing visible radiation to the side-emitting polymeric optical fiber, thereby reducing the aqueous carbon dioxide.

11. The method of claim 10, wherein the aqueous carbon dioxide is free of bubbles.

12. The method of claim 10, wherein reducing the aqueous carbon dioxide yields formic acid.
